(19) United States
(12) Patent Application Publication
(10) Pub. No.: US 2021/0145542 A1
(43) Pub. Date: May 20, 2021

(54) Dental Composite Capsule With Useful Life Indicator

(71) Applicant: David J. Clark, Lakewood, WA (US)

(72) Inventor: David J. Clark, Lakewood, WA (US)

(21) Appl. No.: 16/952,944

(22) Filed: Nov. 19, 2020

Related U.S. Application Data
(60) Provisional application No. 62/938,088, filed on Nov. 20, 2019.

(51) Int. Cl. A61C 5/66; A61K 6/15; A61K 6/884; A61K 6/65; A61C 5/62

(52) U.S. Cl. CPC A61C 5/66; A61K 6/15; A61C 2201/002; A61K 6/65; A61C 5/62; A61K 6/884

(57) ABSTRACT

The present invention relates to a capsule for use in dispensing dental composite. The capsule includes a hollow body having a wall defining an interior space of the body. The body includes a proximal end and a distal end having a dispensing orifice. The wall comprises a polymeric material, and a time-temperature indicating material and/or a temperature indicating material. The capsule includes one or more composite resins positioned in the interior space of the body.

### CROSS-REFERENCES TO RELATED APPLICATIONS

This application claims priority to U.S. Patent Application No. 62/938,088 filed Nov. 20, 2019, which is incorporated herein by reference in its entirety.

### STATEMENT REGARDING FEDERALLY SPONSORED RESEARCH

Not Applicable.

### BACKGROUND OF THE INVENTION

### 1. Field of the Invention

The present invention relates to methods for the restoration of a decayed portion of a tooth or re-restoration of a previously filled tooth, and to composite resin dispensers and capsules that may be used in the methods for the restoration of a decayed portion of a tooth.

### 2. Description of the Related Art

Dental cavities that have spread to the dentin or have undergone cavitation are typically treated by removing the decayed portion of the tooth and thereafter filling the missing tooth structure with a restorative material such as silver (amalgam), white (resin), porcelain, or gold. Cavities that are located adjacent to neighboring teeth are called interproximal cavities.

When treating interproximal cavities, the dentist first removes the decayed portion of the side of the tooth. In order to properly deposit the restorative material on the side of the tooth without undesired leaking of the restorative material beyond the side of the tooth, the dentist places a dental matrix around at least a portion of the tooth. The dental matrix may be a metallic or plastic strip, and when the matrix is placed around at least a portion of the tooth, the matrix acts as a form for the desired shape of the restored tooth.

The Bioclear® brand matrix (available at http://www.bioclearmatrix.com) has advanced dental restoration with an injection molding process (see U.S. Patent Application Publication No. 2008/0064012). In addition, PCT Patent Application Publication No. WO 2016/090246 describes dental composite capsules containing light curable composite resin restorative material. The capsule may be heated in a dental composite dispenser for dispensing heated light curable composite resin into a cavity preparation where the composite resin is light cured. However, certain light curable composite resins may not light cure properly when heated above certain temperatures for extended periods of time.

Therefore, what is needed is a dental capsule with a useful life indicator for the light curable composite resin that has been heated within the capsule.

### SUMMARY OF THE INVENTION

The invention meets the foregoing needs by providing improved dental capsules and dental composite dispensers for the restoration of a decayed portion of a tooth.

An aspect of the invention is a preloaded body (e.g., a capsule) that is designed to be used in conjunction with unheated extrusion or heated extrusion. The capsule may be used with a dental composite dispenser. The capsule can include a hollow body with a wall defining an interior space of the body. The body can include a proximal end and a distal end. The distal end can have a dispensing orifice. The wall of the hollow body can have a polymeric material and a time-temperature indicating material. Preferably, the capsule is a single use, disposable capsule.

A first composite resin is preloaded in the interior space of the body. In one embodiment, the first composite resin has a first viscosity. The first composite resin can comprise a first filler and a light curable resin.

In another embodiment, a second composite resin can be preloaded in the interior space of the body. In this embodiment, a first volume percentage of the first filler in the first composite resin can be greater than a second volume percentage of the second filler in the second composite resin.

In the wall of the capsule, the polymeric material can include polytetrafluoroethylene (commercially available as Teflon), nylon, or other polymer-based materials.

In the embodiment with the first composite resin and the second composite resin, the time-temperature indicating material can be positioned between the first composite resin and the dispensing orifice. The viscosities of the first composite resin and/or second composite resin can be lowered by heating.

In the wall of the capsule, at least a section of the time-temperature indicating material can change color indicating the first composite resin and/or second composite resin have been heated to a predetermined temperature. In addition, the change of color of at least a section of the time-temperature indicating material can indicate that the flowable composite has been heated at a predetermined temperature for a predetermined length of time. The time-temperature indicating material may comprise a polymer and a dye in contact with the polymer, wherein the dye diffuses through the polymer whenever the polymer is at or above the predetermined temperature, and wherein an extent of diffusion of the dye within the polymer corresponds to the predetermined length of time. The polymer may be selected from the group consisting of ethylene-vinyl acetate, polyester, polyethylene, polyethylene/ethylene acrylic acid, polypropylene, polystyrene, polyvinyl chloride, and polyvinylidiene chloride. The dye may be selected from the group consisting of non-toxic dye compounds. The dye may be selected from the group consisting of D&C Red #17, D&C Green #6, D&C Violet #2, D&C Yellow #11, and D&C Green #6.

Another aspect of the invention is a preloaded body (e.g., a capsule) that is designed to be used in conjunction with unheated extrusion or heated extrusion. The capsule may be used with a dental composite dispenser. The capsule can include a hollow body with a wall defining an interior space of the body. The body can include a proximal end and a distal end. The distal end can have a dispensing orifice. The wall of the hollow body can have a polymeric material and a temperature indicating material. At least a section of the temperature indicating material has a first color below a predetermined temperature and changes to a second different color indicating the composite resin has been heated to or above the predetermined temperature. The section of the temperature indicating material changes back reversibly to the first color when the flowable composite has been cooled below the predetermined temperature after having been heated to or above the predetermined temperature.

The temperature indicating material may comprise a polymer blend of a first polymer and a second polymer that is different from the first polymer, and the polymer blend can be single-phase and have the first color at temperatures below the predetermined temperature, and the polymer blend can include separate phases and have a second color different from the first color at temperatures at or above the predetermined temperature. The polymer blend may be selected from the group consisting of polymethyl methacrylate/styrene-acrylonitrile copolymers; polystyrene/polyvinyl methyl ether; poly-ε-caprolactone/styrene-acrylonitrile copolymers; chlorinated rubber/ethylene-vinyl acetate copolymers; polyvinyl chloride/ethylene-vinyl acetate copolymers; poly-ε-caprolactone/polycarbonate; polyvinylidene fluoride/polymethyl methacrylate; polyvinylidene fluoride/polyethyl methacrylate; polyvinylidene fluoride/polymethyl acrylate; polyvinylidene fluoride/polyethyl acrylate; polyphenylene oxide/o-chloro-styrene-p-chlorostyrene copolymers; polystyrene/polycarbonate of tetramethyl-bis-phenol-A; polyvinyl nitrate/polymethyl acrylate; polyvinyl chloride/poly-n-hexylmethacrylate; polyvinyl chloride/poly-n-butyl acrylate, poly-n-propyl acrylate; chlorinated polyethylene/butyl acrylate; polymethyl methacrylate/chlorinated polyethylene; 2-ethylhexyl-methacrylate/isobutyl-methacrylate copolymer; and methyl methacrylate and ethyl acrylate (3:2)/chlorinated rubber copolymer.

Another aspect of the invention is a preloaded body (e.g., a capsule) that is designed to be used in conjunction with unheated extrusion or heated extrusion. The capsule may be used with a dental composite dispenser. The capsule can include a hollow body with a wall defining an interior space of the body. The body can include a proximal end and a distal end. The distal end can have a dispensing orifice. The wall of the hollow body can have a polymeric material and a time-temperature indicating material and a temperature indicating material. A change of color of at least a section of the time-temperature indicating material can indicate that the flowable composite has been heated at a predetermined temperature for a predetermined length of time. At least a section of the temperature indicating material may have a first color below the predetermined temperature and a second different color at or above the predetermined temperature. The section of the temperature indicating material changes back reversibly to the first color when the flowable composite has been cooled below the predetermined temperature after having been heated to or above the predetermined temperature.

Another aspect of the invention is a dental composite dispenser. The dispenser can include a housing with an end section for dispensing composite, and any of the capsules, as described above, in the end section of the housing. The dispenser can also include an electrical power supply and at least one resistive heating element in electrical communication with the power supply. The heating elements can be located in the end section of the housing. A movable piston of the dispenser can engage an inner surface of the body. Movement of the piston toward the dispensing orifice extrudes the first composite resin and/or second composite resin from the dispensing orifice of the capsule.

Thus, the invention includes the manufacture and placement of flowable composite dental filling material and/or paste composite dental filling material into preloaded unidose type capsules. The capsules may possess reduced tip orifice size through which the extrusion of the flowable composite and/or the paste composite through the smaller dispensing orifice is dependent on alteration of the physical characteristics of the paste composite from heat and/or extreme pressure or other means by which the physical properties of the paste composite are altered to allow increased flowability (such as vibration, ultrasonic energy, microwaves, or similar physical and thermal energies). The composite within the capsule can be varied in viscosity by heating the composite within the capsule. The temperature and heating of the composite can be monitored by a temperature-indicating material while the flowable composite is heated. Once the flowable composite is heated to a predetermined temperature, at least a section of the temperature indicating material indicates the temperature with a change of color. In addition, a change of color of at least a section of a time-temperature indicating material can indicate that the flowable composite has been heated at a predetermined temperature for a predetermined length of time. The change of color prevents the flowable composite from being overheated.

Heating of the composite and changing the small dispensing orifice with a reduced tip orifice size allows for: (i) the direct placement into cavities that are smaller than the circumference of traditional paste capsule tip orifice size, (ii) an injection molded composite technique which requires deeper insertion of the capsule tip into the cavity preparation, and (iii) use with anatomic and pre-curved matrices that impede the insertion of larger, traditional tip orifices. The time-temperature indicating material and/or the temperature indicating material in the capsule wall provides a useful life indicator for composite resin that has heated above certain temperatures for extended periods of time.

The features, aspects, and advantages of the present invention will become better understood upon consideration of the following detailed description, drawings and appended claims.

### BRIEF DESCRIPTION OF THE DRAWINGS

FIG. 1 shows a vertical cross-sectional view of a dental capsule according to one embodiment of the invention.

FIG. 2 is an enlarged side view of the capsule of FIG. 1.

FIG. 2A is a vertical cross-sectional view of the capsule of FIG. 1 showing the position of plunger and the pre-loaded composite resin filling materials.

FIG. 3A is a side view of another capsule according to another embodiment of the invention.

FIG. 3B is a side view of yet another capsule according to yet another embodiment of the invention.

FIG. 4 is a side view of another embodiment of a composite dispenser according to the invention from which composite resin filling materials are dispensed.

FIG. 5 is a top perspective view of a composite warmed with composite dispensers of FIG. 4 according to the invention.

Like reference numerals will be used to refer to like parts from Figure to Figure in the following description of the drawings.

### DETAILED DESCRIPTION OF THE INVENTION

The invention provides improved methods and dental composite capsules for the restoration of a decayed portion of a tooth.

In an example method according to the invention for the restoration of a tooth, the dentist locates a tooth having a cavity. A hollow cavity preparation is prepared in a tooth. The tools and techniques for forming the hollow cavity preparation are well known in the art and therefore will not be explained further.

In order to properly deposit the restorative material on the tooth without undesired leaking of the restorative material beyond the tooth, the dentist may place a dental matrix around at least a portion of the tooth. A sectional anatomic translucent dental matrix may be placed on the tooth. When the matrix is placed around at least a portion of the tooth, the matrix acts as a form for the desired shape of the restored tooth.

The cavity preparation in the tooth is then etched with liquid and/or gel phosphoric acid. The cavity preparation in the tooth is then rinsed and dried. A lightly filled or unfilled light curable resin tooth bonding agent is then applied to the tooth covering the entire cavity preparation. The resin tooth bonding agent is not light cured at this point. Resin tooth bonding agents improve composite to enamel and/or dentin bonding.

A light curable flowable composite resin is then injected from a dental capsule according to the invention directly into the pool of resin tooth bonding agent. The light curable flowable composite and resin tooth bonding agent are not light cured at this point. Generally, light curable flowable composite resins contain 20-25 percent less filler in the light curable polymeric material than nonflowable paste materials. Light curable resins are preferred as light cured resins are more color stable than chemically cured resins.

A light curable paste composite resin is then extruded from the same dental capsule according to the invention into the pool of flowable composite resin and resin tooth bonding agent. The next steps are burnishing, carving the anatomy and carving excess composite. There is no need to use a condenser or plugger.

The filled cavity preparation is then cured using a curing light such as high intensity light emitting diode (LED) lights, plasma-arc curing lights, halogen lights, and laser lights. The matrix is then removed, and the restored tooth is polished with discs, strips, and rubber tipped and carbide burs.

One version of the invention provides a capsule for use in a dental composite dispenser. The capsule includes a hollow body having a wall defining an interior space of the body. The body includes a proximal end and a distal end having a dispensing orifice. The wall can have a polymeric material, and a time-temperature indicating material and/or a temperature indicating material. A first composite resin positioned in the interior space of the body. The first composite resin has a first viscosity and can be a paste composite, or a flowable composite.

In this version of the capsule, the first composite resin is a flowable composite, and the viscosity of the first composite resin can be lowered by heating.

The first composite resin can be a bulk flowable composite, or a resin modified glass ionomer composite. The capsule, the capsule is a single use, disposable capsule. As the viscosity of the flowable composite is lowered by heating, a color change of at least a section of the temperature indicating material indicates that the flowable composite has reached a predetermined temperature by a change in color. In addition, the change of color of at least a section of the time-temperature indicating material can indicate that the flowable composite has been heated at a predetermined temperature for a predetermined length of time.

Another version of the invention provides a dental composite dispenser comprising a housing including an end section for dispensing composite; and any version of the capsules described herein in the end section of the housing. The dispenser can include an electrical power supply; and at least one resistive heating element in electrical communication with the power supply. Each heating element is located in the end section of the housing. The dispenser can comprise a movable piston engaging the inner surface of the body, wherein movement of the piston toward the dispensing orifice sequentially extrudes the composite resin(s) from the dispensing orifice of the capsule.

Another version of the invention provides a method for the restoration of a tooth. The method includes the steps of: (a) removing a portion of the tooth to form a cavity preparation; (b) sequentially extruding composite resins into the cavity preparation from the dispensing orifice of one version of the capsule; and (c) simultaneously light curing the composite resin contained in the cavity preparation. The method can further comprise placing a light-curable resin tooth bonding agent into the cavity preparation. The method can further comprise surrounding the cavity preparation with a matrix. The method can include the step of loading the capsule into an end section of a housing of a dental composite dispenser and sequentially extruding the second composite resin and the first composite resin from the dispensing orifice of the capsule.

In the method, the dental composite dispenser can comprise an electrical power supply, and at least one resistive heating element in electrical communication with the power supply wherein each heating element is located in the end section of the housing. The dental composite dispenser can comprise a movable piston engaging the inner surface of the body, and movement of the piston toward the dispensing orifice sequentially extrudes the composite resin from the dispensing orifice. The method can include the step of extruding a light-curable resin tooth bonding agent from the capsule into the cavity preparation. The method can include the step of extruding a light-curable resin tooth bonding agent from the capsule into the cavity preparation and breaking a frangible barrier positioned in the interior space of the body between the second composite resin and the light-curable resin tooth bonding agent. The method can include the step of extruding a light-curable resin tooth bonding agent from a second capsule into the cavity preparation. In the method, movement of a second piston of the dental composite dispenser toward a dispensing orifice of the second capsule extrudes the light-curable resin tooth bonding agent from the dispensing orifice of the second capsule.

The dental composite dispenser can be placed into a composite warmer to heat the composite resin within the capsule. A color change of at least a section of a temperature indicating material within the wall of the capsule can indicate the composite resin has been heated to a predetermined temperature. In addition, the change of color of at least a section of a time-temperature indicating material can indicate that the flowable composite has been heated at a predetermined temperature for a predetermined length of time.

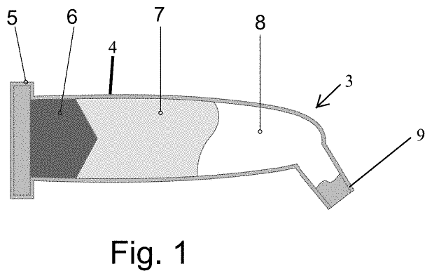

Turning now to FIG. 1, there is shown a dental capsule 3 according to one non-limiting embodiment of the invention. The capsule 3 has a wall 4 defining a hollow body 5. An interior space of the body 5 contains a plunger 6 in contact with a high viscosity dental composite resin 7, which is in contact with a lower viscosity composite resin 8, which is adjacent a dispensing orifice 9. The plunger 6 is positioned between the composite resin 7,8 and the proximal end of the body 5. The composite resins 7, 8 may have different opacities. The composite resins 7, 8 may have different colors. The composite resins 7, 8 may be the same composite resin. The viscosity of the composite resin 7,8 can be lowered by heating the capsule 3. The composite resins 7, 8 may be in contact before use of the capsule 3 in a dental composite dispenser as no barrier need be positioned between the resin 7 and the resin 8. Although the use of a high viscosity dental composite resin 7 and a lower viscosity composite resin 8 is advantageous, the interior space of the body 5 may contain a single dental composite resin.

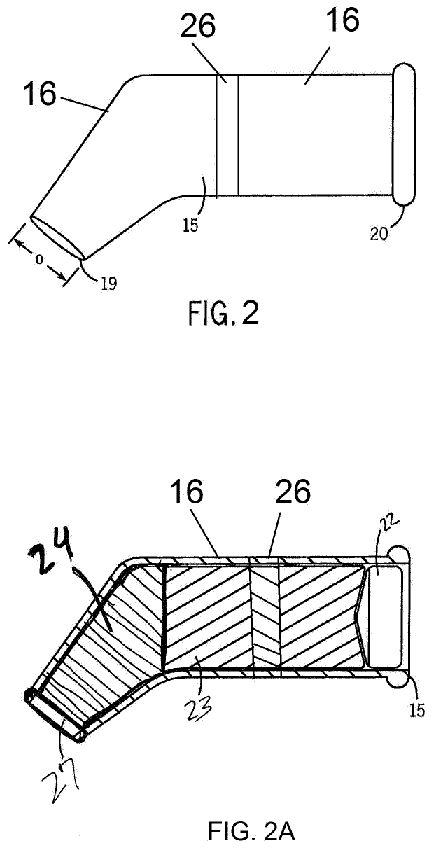

FIG. 2 is a side view, close up of the capsule 15 having a dispensing orifice 19 with inside diameter O (which can be 2.5 millimeters) and a proximal end 20. The wall 4 of the capsule 15 having a polymeric material 16 and a time-temperature indicating material 26. FIG. 2A is a cross-sectional view of the capsule 15 showing the position of plunger 6 of the dispenser gun 11 and a pre-loaded first (paste) composite resin filling material 23 and second (flowable) composite resin filling material 24 which both move forward by way of a rear sliding disc 22 which is contacted by the plunger of the dispenser gun 11. The wall of the capsule contains a polymeric material 16 and a time-temperature indicating material 26. The time-temperature indicating material 26 is positioned between the composite resin 7,8 and the dispensing orifice 9. An end cap 27 covers the dispensing orifice 19 before use of the capsule 15. Although the use of a first (paste) composite resin filling material 23 and a second (flowable) composite resin filling material 24 is advantageous, the interior space of the capsule may contain a single dental composite resin.

The polymeric material 16 is made of polytetrafluoroethylene, nylon, polyethylene, polypropylene, nylon, acrylonitrile-butadiene-styrene (ABS) or other medically suitable polymeric material.

At least a section of the time-temperature indicating material 26 changes color indicating the composite resin 7, 8 has been heated to a predetermined temperature. In addition, the change of color of at least a section of the time-temperature indicating material 26 can indicate that the flowable composite has been heated at a predetermined temperature for a predetermined length of time. The time-temperature indicating material 26 may comprise a polymer and a dye in contact with the polymer, wherein the dye diffuses through the polymer whenever the polymer is at or above the predetermined temperature, and wherein an extent of diffusion of the dye within the polymer corresponds to the predetermined length of time. The polymer may be selected from the group consisting of ethylene-vinyl acetate, polyester, polyethylene, polyethylene/ethylene acrylic acid, polypropylene, polystyrene, polyvinyl chloride, and polyvinylidiene chloride. The dye may be selected from the group consisting of non-toxic dye compounds. The dye may selected from the group consisting of D&C Red #17, D&C Green #6, D&C Violet #2, D&C Yellow #11, and D&C Green #6. U.S. Pat. No. 5,476,792, which is incorporated by reference, describes materials suitable for use in the time-temperature indicating material 26.

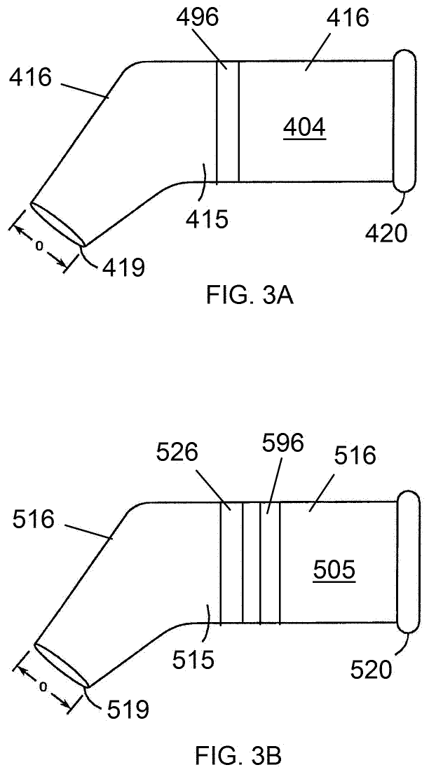

FIG. 3A is a side view of another capsule 415 according to another embodiment of the invention. The capsule 415 has a dispensing orifice 419 with inside diameter O (which can be 2.5 millimeters) and a proximal end 420. The wall 404 of the capsule 415 comprises a polymeric material 416 and a temperature indicating material 496. The capsule 415 may contain one or more composite resin filling materials in the interior space of the capsule. At least a section of the temperature indicating material 496 changes color indicating the composite resin has been heated to a predetermined temperature, for example, 155° F. If the resin temperature cools down below the predetermined temperature, the color change of the temperature indicating material 496 is reversible. The temperature indicating material 496 may comprise a polymer blend of a first polymer and a second polymer that is different from the first polymer. At temperatures below the predetermined temperature, the polymer blend is single-phase and has a first color, and at temperatures at or above the predetermined temperature, the polymer blend includes separate phases and has a second color different from the first color. As used herein, the “first color” may also refer to a transparent appearance or an opaque appearance, and the “second color” may also refer to a transparent appearance or an opaque appearance. Non-limiting example polymer blends useful for the temperature indicating material 496 include polymethyl methacrylate/styrene-acrylonitrile copolymers; polystyrene/polyvinyl methyl ether; poly-ε-caprolactone/styrene-acrylonitrile copolymers; chlorinated rubber/ethylene-vinyl acetate copolymers; polyvinyl chloride/ethylene-vinyl acetate copolymers; poly-ε-caprolactone/polycarbonate; polyvinylidene fluoride/polymethyl methacrylate; polyvinylidene fluoride/polyethyl methacrylate; polyvinylidene fluoride/polymethyl acrylate; polyvinylidene fluoride/polyethyl acrylate; polyphenylene oxide/o-chloro-styrene-p-chlorostyrene copolymers; polystyrene/polycarbonate of tetramethyl-bis-phenol-A; polyvinyl nitrate/polymethyl acrylate; polyvinyl chloride/poly-n-hexylmethacrylate; polyvinyl chloride/poly-n-butyl acrylate, poly-n-propyl acrylate; chlorinated polyethylene/butyl acrylate; polymethyl methacrylate/chlorinated polyethylene; 2-ethylhexyl-methacrylate/isobutyl-methacrylate copolymer; and methyl methacrylate and ethyl acrylate (3:2)/chlorinated rubber copolymer. U.S. Pat. No. 4,772,506, which is incorporated by reference, describes materials suitable for use in the temperature indicating material 496.

FIG. 3B is a side view of another capsule 515 according to another embodiment of the invention. The capsule 515 has a dispensing orifice 519 with inside diameter O (which can be 2.5 millimeters) and a proximal end 520. The wall 505 of the capsule 515 comprises a polymeric material 516 and a time-temperature indicating material 526 and a temperature indicating material 596. The capsule 515 may contain one or more composite resin filling materials in the interior space of the capsule.

At least a section of the time-temperature indicating material 526 changes color indicating the composite resin has been heated to a predetermined temperature. In addition, the change of color of at least a section of the time-temperature indicating material 526 can indicate that the flowable composite has been heated at a predetermined temperature for a predetermined length of time. The time-temperature indicating material 526 may comprise the same materials described above for the time-temperature indicating material 26.

At least a section of the temperature indicating material 596 changes color indicating the composite resin has been heated to a predetermined temperature, for example, 155° F. If the resin temperature cools down below the predetermined temperature, the color change of the temperature indicating material 596 is reversible. The temperature indicating material 596 may comprise the same materials described above for the temperature indicating material 496.

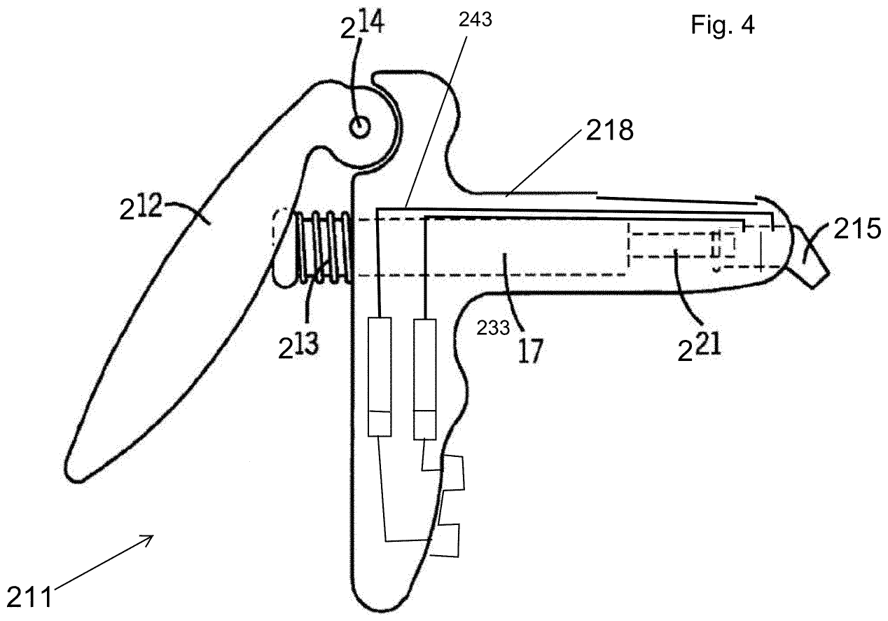

In FIG. 4, there is shown a non-limiting example dental composite dispenser of the invention. The dental composite dispenser 211 has a capsule 215 in which resin composite filling material(s) are loaded. A piston 217 in the dispenser housing 218 drives a rubber plunger 221 which in turn presses the composite resin filling material. The handle 212 of the dispenser 211 is pressed and compresses a spring 213. A hinge 214 allows rotation of the handle 212 to press the piston 217.

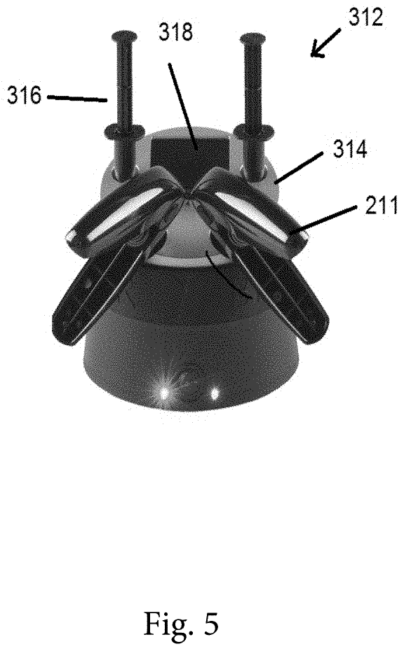

In FIG. 5, there is shown a composite warmer 312 having a housing 314 for the dental composite dispenser 211. The capsule 215 within the composite dispenser 211 is heated in the composite warmer 312. Capsules 415 or 515 may also be used. The composite warmer 312 is creates void free injection molded composites. Heating the composite fillings within the capsule 215 (or capsule 415 or capsule 515) with the composite warmer 312 warms the fillings up to a constant temperature causing a change in the viscosity of the fillings. Once the fillings have been warmed to a predetermined temperature, for example, 155° F., at least a section of the time-temperature indicating material 26 for capsule 215 (or the temperature indicating material 496 for capsule 415 or temperature indicating material 596 for capsule 515) will show a change in color to indicate the temperature of the resin in the capsule 215 (or 415 or 515). In addition, another change of color of at least another section of the time-temperature indicating material 26 for capsule 215 (or time-temperature indicating material 526 for capsule 515) indicates that the flowable composite has been heated at a predetermined temperature for a predetermined length of time. The composite warmer 312 has ample space for all the composite needed for restorative cases. The layout of the warmer 312 facilitates heating of two composite dispensers 211 with two flowable syringes 316 and a compartment 318 for storage of up to six extra composite capsules 215. The composite reaches its optimum working temperature (approximately 155° F.) quickly, due to the large anodized aluminum heat transfer top. The ergonomic design of the composite warmer 312 and its heating slots in which the composite dispensers 211 are placed, ensures that the composite dispensers 211 and flowable syringes 316 are easy to handle when placing into and removing from the unit. The removable heat transfer top is made of the highest quality autoclavable materials. The wall of each of the two flowable syringes 316 (which may also be referred to as capsules herein) can include a time-temperature indicating material as described above and a temperature indicating material as described above.

The paste composite resin filling material and the flowable composite resin filling material may include a polymerizable (e.g., light curable) resin and a filler. Non-limiting examples of suitable resins include acrylate resins, methacrylate resins, and silorane-based resins. Non-limiting examples of suitable fillers include silica, silicate glass, quartz, barium silicate, strontium silicate, barium borosilicate, strontium borosilicate, borosilicate, lithium silicate, lithium alumina silicate, amorphous silica, calcium phosphate, alumina, zirconia, tin oxide, and titania. The paste composite can include greater than 30% by volume filler, or greater than 40% by volume filler, or greater than 50% by volume filler, or greater than 60% by volume filler, or greater than 70% by volume filler, or greater than 80% by volume filler, or greater than 90% by volume filler. Preferably, the dental restorative material (a combination of the first paste composite resin material and the second flowable composite resin filling material) has a volume shrinkage of 3% or less upon curing, More preferably, the dental restorative material has a volume shrinkage of 2% or less upon curing. Most preferably, the dental restorative material has a volume shrinkage of 1% or less upon curing.

Various dental matrices may be used with the invention. Each matrix can be tooth specific, or the matrix can be tooth type specific, or the matrix can be tooth surface specific. By “tooth specific” it is meant that the matrix is configured to conform to the shape of the outer surface of the specific natural tooth being restored such as (without limitation) an upper left central incisor. By “tooth type specific” it is meant that the matrix is configured to conform to the shape of the outer surface of the specific type of natural tooth being restored such as (without limitation) an upper incisor. By “tooth surface specific” it is meant that the matrix is configured to conform to the shape of the outer surface of the specific natural tooth surface being restored such as (without limitation) an upper left incisor mesial surface.

Thus, the invention provides methods for the restoration of a decayed portion of a tooth or re-restoration of a previously filled tooth, and also provides dental capsules and composite resin dispensers that may be used in the methods for the restoration of a decayed portion of a tooth

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A capsule for use in dispensing dental composite, the capsule comprising:
a hollow body comprising a wall defining an interior space of the body, the body including a proximal end and a distal end having a dispensing orifice, the wall comprising a polymeric material and a time-temperature indicating material; and
a composite resin positioned in the interior space of the body.

2. The capsule of claim 1, wherein at least a section of the time-temperature indicating material changes color indicating the composite resin has been heated to a predetermined temperature.

3. The capsule of claim 1, wherein at least a section of the time-temperature indicating material changes color indicating that the flowable composite has been heated at a predetermined temperature for a predetermined length of time.

4. The capsule of claim 3, wherein:
the time-temperature indicating material comprises a polymer and a dye in contact with the polymer, and
the dye diffuses through the polymer whenever the polymer is at or above the predetermined temperature, and
wherein an extent of diffusion of the dye within the polymer corresponds to the predetermined length of time.

5. The capsule of claim 4, wherein:
the polymer is selected from the group consisting of ethylene-vinyl acetate, polyester, polyethylene, polyethylene/ethylene acrylic acid, polypropylene, polystyrene, polyvinyl chloride, and polyvinylidiene chloride.

6. The capsule of claim 1, wherein:
the composite resin comprises a first filler and a light curable resin, and the composite resin has a first viscosity, and
the capsule further comprises a second composite resin positioned in the interior space of the body, the comprising a second filler and a second light curable resin and
a first volume percentage of the first filler in the composite resin is greater than a second volume percentage of the second filler in the second composite resin.

7. The capsule of claim 6, wherein the time-temperature indicating material is positioned between the composite resin and the dispensing orifice.

8. The capsule of claim 1, wherein the viscosity of the composite resin can be lowered by heating.

9. A dental composite dispenser comprising:
a housing including an end section for dispensing composite; and
the capsule of claim 1 in the end section of the housing.

10. A capsule for use in dispensing dental composite, the capsule comprising:
a hollow body comprising a wall defining an interior space of the body, the body including a proximal end and a distal end having a dispensing orifice, the wall comprising a polymeric material and a temperature indicating material; and
a composite resin positioned in the interior space of the body.

11. The capsule of claim 10, wherein at least a section of the temperature indicating material has a first color below a predetermined temperature and changes to a second color indicating the composite resin has been heated to or above the predetermined temperature.

12. The capsule of claim 11, wherein the section of the temperature indicating material changes back to the first color indicating that the flowable composite has been cooled below the predetermined temperature after having been heated to or above the predetermined temperature.

13. The capsule of claim 12, wherein:
the temperature indicating material comprises a polymer blend of a first polymer and a second polymer that is different from the first polymer, and
the polymer blend is single-phase and has a first color at temperatures below the predetermined temperature, and
the polymer blend includes separate phases and has a second color different from the first color at temperatures at or above the predetermined temperature.

14. The capsule of claim 13, wherein the polymer blend is selected from the group consisting of polymethyl methacrylate/styrene-acrylonitrile copolymers; polystyrene/polyvinyl methyl ether; poly-ε-caprolactone/styrene-acrylonitrile copolymers; chlorinated rubber/ethylene-vinyl acetate copolymers; polyvinyl chloride/ethylene-vinyl acetate copolymers; poly-ε-caprolactone/polycarbonate; polyvinylidene fluoride/polymethyl methacrylate; polyvinylidene fluoride/polyethyl methacrylate; polyvinylidene fluoride/polymethyl acrylate; polyvinylidene fluoride/polyethyl acrylate; polyphenylene oxide/o-chloro-styrene-p-chlorostyrene copolymers; polystyrene/polycarbonate of tetramethyl-bis-phenol-A; polyvinyl nitrate/polymethyl acrylate; polyvinyl chloride/poly-n-hexylmethacrylate; polyvinyl chloride/poly-n-butyl acrylate, poly-n-propyl acrylate; chlorinated polyethylene/butyl acrylate; polymethyl methacrylate/chlorinated polyethylene; 2-ethylhexyl-methacrylate/isobutyl-methacrylate copolymer; and methyl methacrylate and ethyl acrylate (3:2)/chlorinated rubber copolymer.

15. The capsule of claim 10, wherein the wall further comprises a time-temperature indicating material.

16. The capsule of claim 15, wherein at least a section of the time-temperature indicating material changes color indicating the composite resin has been heated to a predetermined temperature.

17. The capsule of claim 15, wherein at least a section of the time-temperature indicating material changes color indicating that the flowable composite has been heated at a predetermined temperature for a predetermined length of time.

18. The capsule of claim 17, wherein:
the time-temperature indicating material comprises a polymer and a dye in contact with the polymer, and
the dye diffuses through the polymer whenever the polymer is at or above the predetermined temperature, and
wherein an extent of diffusion of the dye within the polymer corresponds to the predetermined length of time.

19. The capsule of claim 10, wherein:
the composite resin comprises a first filler and a light curable resin, and the composite resin has a first viscosity, and
the capsule further comprises a second composite resin positioned in the interior space of the body, the comprising a second filler and a second light curable resin and
a first volume percentage of the first filler in the composite resin is greater than a second volume percentage of the second filler in the second composite resin.

20. A dental composite dispenser comprising:
a housing including an end section for dispensing composite; and
the capsule of claim 10 in the end section of the housing.
